(19) United States
(12) Patent Application Publication
(10) Pub. No.: US 2001/0049534 A1
(43) Pub. Date: Dec. 6, 2001

(54) Device for inserting an aortic endoprosthesis

(75) Inventor: Mario Lachat, Zurich (CH)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: FUMEDICA Intertrade AG

(21) Appl. No.: 09/882,448

(22) Filed: Jun. 15, 2001

Related U.S. Application Data
(63) Continuation of application No. PCT/EP99/09827, filed on Dec. 11, 1999.

(30) Foreign Application Priority Data
Dec. 16, 1998 (GB) 298 22 381.3

(51) Int. Cl. A61F 2/06

(52) U.S. Cl. 606/108; 623/1.11

(57) ABSTRACT

Known devices for inserting aortic endoprostheses include a guide wire with which the opening of the main prosthesis, which is already in the aneurysm, has to be probed for insertion of the extension. This is unsafe and time-consuming. In the present device a second guide wire for the extension is alignably fixed to the first guide wire for the main prosthesis, so that the opening for receiving the extension can be quickly and safely probed from the interior.

### CROSS-REFERENCE TO RELATED APPLICATION

This application is a continuation of International Application No. PCT/EP99/09827, filed Dec. 11, 1999, the disclosure of which is incorporated herein by reference.

### BACKGROUND OF THE INVENTION

The invention concerns a device for inserting aortic endoprostheses, in particular in the treatment of abdominal aorta aneurysms.

For the minimally invasive surgical treatment of an abdominal aorta aneurysm, it is known to implant an endoprosthesis in the form of a coated stent which relieves the wall of the aorta of the load of the blood pressure in the region of the aneurysm 2 (FIG. 1) and prevents further dilation and tearing thereof. That stent must project upwardly and downwardly beyond the region of the aneurysm 2, that is to say it must begin at the top in the undilated region of the aorta 1 and lead into both pelvic arteries 3, 4. The stent is usually composed of two parts, wherein a main prosthesis 5 covers the aorta and the adjoining branch of a pelvic artery 3 and an extension 6 covers the branch of the other pelvic artery 4.

In the insertion procedure, the arteria femoralis is first punctured on one side, for example on the right, then a guide wire and subsequently a sleeve are pushed into the aorta 1, and finally the main prosthesis is liberated by withdrawing the sleeve. Thereafter, a further guide wire 7 (FIG. 2) is pushed through the left arteria femoralis to the aorta 1, in which case the short limb of the main prosthesis 5 has to be probed with the end of the further guide wire. This process is difficult and often time-consuming. Thereafter, the extension 6 is placed on the left-hand side using the further guide wire, so that it connects the main prosthesis 5 to the pelvic artery 4, which is contra-lateral to the main prosthesis.

### BRIEF SUMMARY OF THE INVENTION

An object of the invention is to provide a device with which the extension can be inserted more easily, more quickly and more safely. This object is attained by a device for inserting a vessel endoprosthesis into an abdominal aorta aneurysm, including:

a first straight elastic guide wire;

a second straight elastic guide wire which is about 10 to 50 cm, preferably about 10 to 30 cm, long and which is fixed with its proximal end to the first guide wire in such a way that it forms with the proximal portion thereof in the stress-free condition an angle of between 20 and 40 degrees, wherein the fixing point is about 10 to 40 cm, preferably about 20 to 40 cm, from the proximal end of the first guide wire; and

an aligning member which is displaceable along the first guide wire and which can assume a first position in which both guide wires are stress-free and a second position in which both guide wires are aligned approximately parallel to each other.

The terms proximal and distal are related here to the patient. Accordingly, the leading end of the guide wire is referred to as the proximal end.

In the first position of the aligning member, the second guide wire is spread away from the first guide wire by about 20 to 40 degrees. This condition obtains after the proximal end of the first guide wire has been introduced into the main prosthesis when already inserted into the aorta. Slight withdrawal of the device according to the invention provides in a simple manner that the distal end of the second guide wire passes through the short end of the main prosthesis and can be engaged with a further guide wire which is introduced by way of the contra-lateral arteria femoralis and which is provided at the end with a wire loop (‘lasso’). Thereafter, the extension can be inserted along that guide wire. In the first position the device according to the invention can also be inserted into the aorta from the exterior through a catheter tube, in which case the catheter provides for parallel guidance of the two wires.

In the second position of the aligning member, the first and second guide wires are approximately parallel. In that position relative to each other, they can be retracted from the region of the aortic aneurysm through the arteriae iliaca and femoralis.

The material and the diameter of the two connected guide wires correspond to what is usual in accordance with the above-described state of the art.

The fixing of the second guide wire to the first can be effected by laser welding.

Advantageously, the ends of the guide wires are curved in an arc of about 90 to 210 degrees with a radius of about 2 to 20 mm, preferably about 5 to 20 mm. This avoids vessel lesions upon insertion of the device.

In another advantageous embodiment the catheter tube serving as an aligning member is closed at the proximal end and is provided with a conical rounded-off tip. In this case, the proximal ends of the guide wires are not curved.

Unnecessary blood loss can be avoided by arranging a hemostatic valve at the distal end of the catheter tube.

While in accordance with the known process the procedure involved in probing the main prosthesis and insertion of the extension can last for up to 30 minutes, a reduction of that period to about 5 minutes is possible with the new device.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a diagrammatic view around the area of the aneurysm with an inserted two-part endoprosthesis;

FIG. 2 is a diagrammatic view similar to FIG. 1, showing the probing procedure in accordance with the prior art;,

FIG. 3 is a schematic perspective view of a device according to a first embodiment of the invention, shown in the first position of the aligning member;

FIG. 4 is a schematic view of the device according to FIG. 3, shown in the second position of the aligning member;

FIG. 5 is a schematic perspective view of a device according to a second embodiment of the invention, shown in the first position of the aligning member;

FIG. 6 is a schematic view of the device according to FIG. 5, shown in the second position of the aligning member;

FIG. 7 is a diagrammatic view of the device illustrated in FIG. 3, shown in the operational area;

FIG. 8 is a diagrammatic view of the device illustrated in FIGS. 3 and 7, shown in the second position of the aligning member in the operational area;

FIG. 9 is a diagrammatic view of a third embodiment of the device according to the invention, shown in the second position of the aligning member; and

FIG. 10 is a detail view from FIG. 9, shown in section on an enlarged scale.

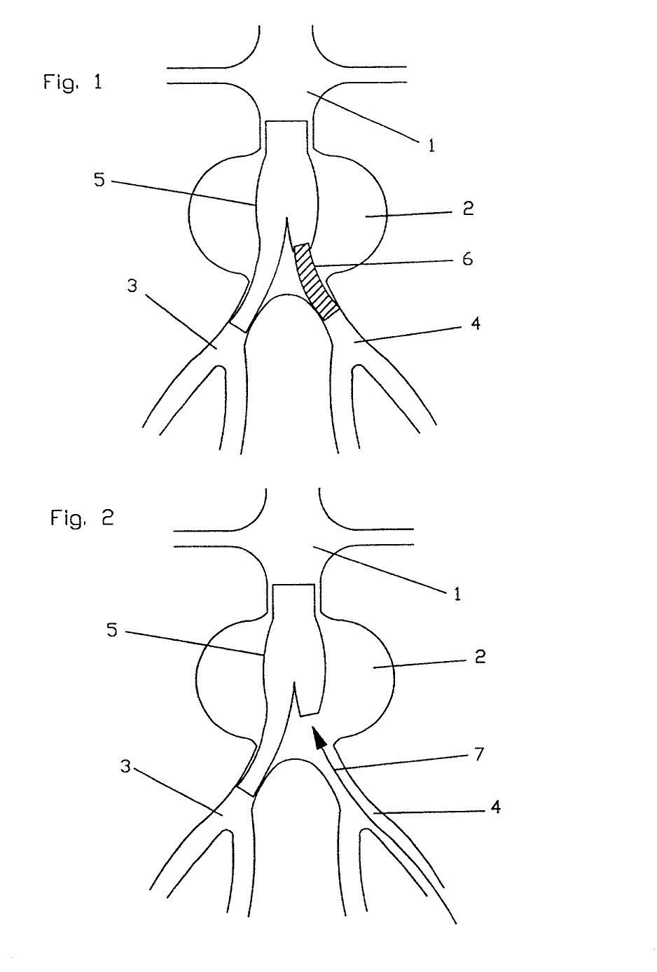

### DETAILED DESCRIPTION OF THE INVENTION

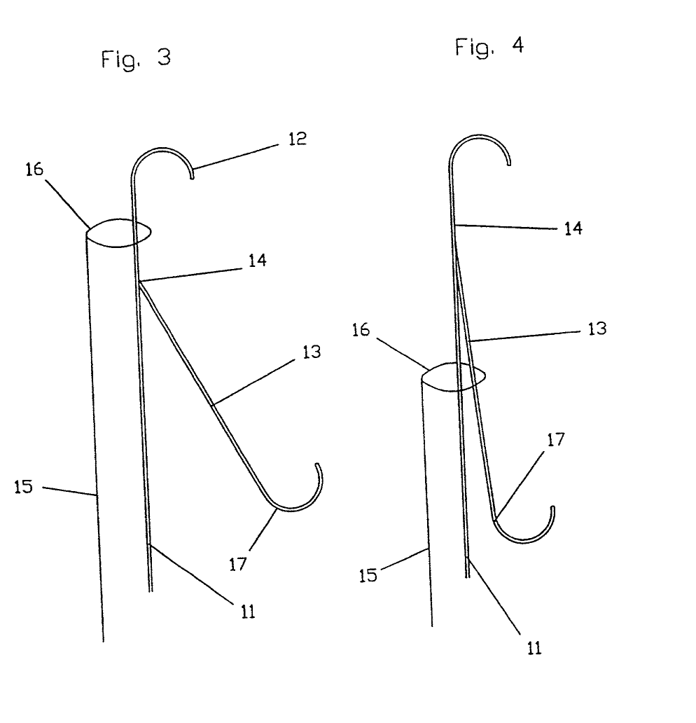

The first guide wire 11 of the device of the first embodiment, which is shown in FIG. 3, is preferably about 130 to 280 cm in length. At the proximal end 12, that is to say the leading end in the insertion procedure, guide wire 11 has a curvature. A second guide wire 13, which is preferably about 10 to 50 cm, most preferably about 10 to 30 cm, long, is fixed with its proximal end attached to the wire 11 in such a way that it forms therewith an angle of about 20 to 40 degrees. The fixing point 14 is about 10 to 40 cm, preferably about 20 to 40 cm, away from the proximal end 12 of the first guide wire 11. A wire loop 16 on a pull wire 15 extends around the wire 11 in proximal relationship to the fixing point. The wire loop preferably has a diameter of about 5 to 10 mm. The pull wire 15 is so long that its distal end remains accessible to the operator.

FIG. 4 shows the device of the first embodiment in the second position of the wire loop 16 which acts as an aligning member. The loop 16 is now disposed in distal relationship with the fixing point 14, and it extends around both guide wires in the proximity of the distal end 17 of the second guide wire 13 and thereby holds these in approximately parallel alignment. In this position the device can be withdrawn from the arteria femoralis without difficulty.

Referring to FIG. 5, a device according to the second embodiment is shown with the aligning member in the first position. Here, a catheter tube 18 acts as the aligning member and has a lateral opening 19, through which the second guide wire 13 can pass in a stress-free condition. The catheter tube 18 is of such a length that its distal end remains accessible even after insertion into the aorta. The opening 19 is preferably about 5 to 20 cm away from the proximal end of the catheter tube.

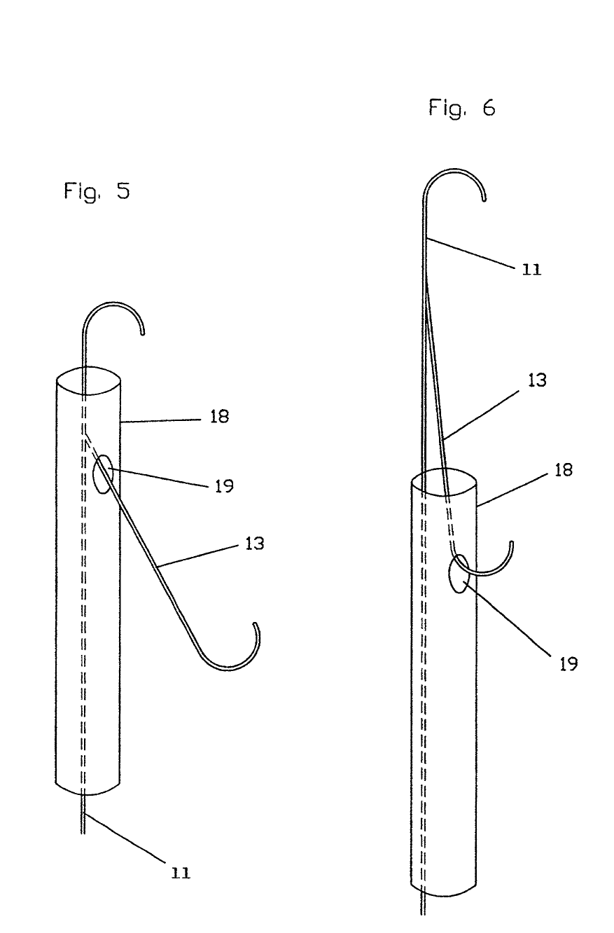

FIG. 6 shows the same device of the second embodiment in the second position of the catheter tube 18. The catheter tube 18 has now been withdrawn relative to the guide wires 11, 13, so that it holds them in approximately parallel alignment in its interior. In this position the device can be withdrawn from the arteria femoralis without difficulty.

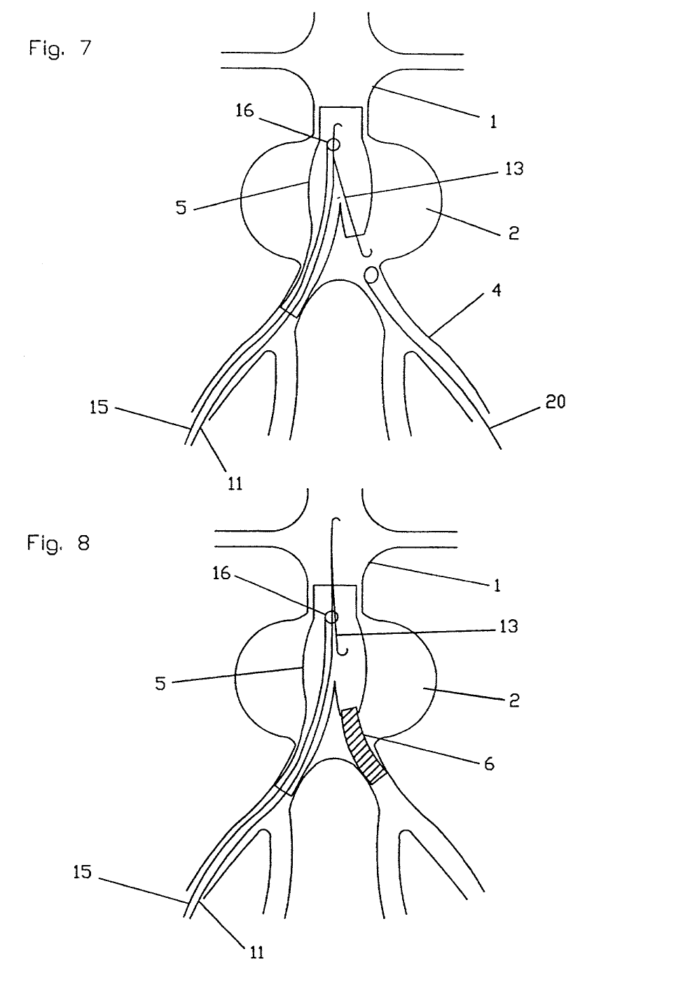

FIG. 7 shows the device of the first embodiment in the first position of the wire loop 16 fixed to the pull wire 15, after insertion into the abdominal aorta 1 and after slight withdrawal thereof. The second guide wire 13 is spread away from the first guide wire 11 and issues with its distal end out of the main prosthesis. That end can now be easily engaged by a lasso 20, on which finally the extension can be inserted and securely placed in the main prosthesis 5 and the pelvic artery 4.

FIG. 8 shows the device of the first embodiment in the second position of the wire loop 16. After insertion of the extension 6, the lasso 20 was released from the second guide wire 13, the guide wire 11 was somewhat further inserted, and the wire loop 16 was drawn downwardly over the fixing point 14 by means of the pull wire 15. As a result, the first and second guide wires are approximately parallel and can be easily drawn out through the arteria femoralis.

The device of the second embodiment can also be used in a similar manner to that shown in FIGS. 7 and 8.

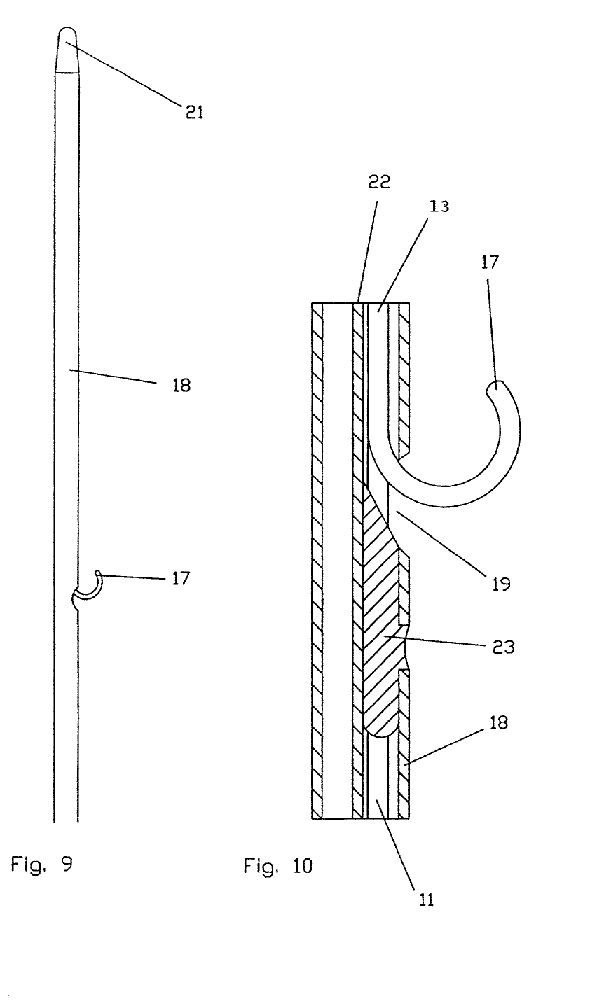

FIG. 9 shows a third embodiment of the device according to the invention. The catheter tube 18 is here closed at the proximal end by a conically rounded-off tip 21. In this case, the proximal end of the first guide wire is not curved.

FIG. 10 is a view in section on an enlarged scale of the region around the exit opening 19 of the device shown in FIG. 9. Shown here are the first guide wire 11 and the second guide wire 13 which is disposed in the upper region thereabove. In order to ensure reliable guidance for the wires, the device also has a separating wall 22 in the catheter tube 18 and an exit guidance plug 23 for the distal end 17 of the second guide wire 13.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A device for inserting a vessel endoprosthesis into an abdominal aorta aneurysm, comprising:
a first straight elastic guide wire;
a second straight elastic guide wire having a length of about 10 to 50 cm and a fixing point with its proximal end to the first guide wire, such that in a stress-free condition the second guide wire forms with a portion of the proximal of the first guide wire an angle of about 20 to 40 degrees, wherein the fixing point is about 10 to 40 cm from the proximal end of the first guide wire; and
an aligning member which is displaceable along the first guide wire and which can assume a first position in which both first and second guide wires are stress-free and a second position in which both first and second guide wires are aligned approximately parallel to each other.

2. The device as set forth in claim 1, wherein the aligning member comprises a pull wire and a wire loop, which in the first position extends proximally around the first guide wire and in the second position extends around both guide wires distally in relation to the fixing point.

3. The device as set forth in claim 1, wherein the aligning member is a catheter tube which encloses the first guide wire and which has a lateral opening permitting the second guide wire to pass therethrough.

4. The device as set forth in claim 1, wherein ends of the first and second guide wires are curved with a radius of about 2 to 20 mm through an arc of about 90 to 210 degrees.

5. The device as set forth in claim 3, wherein the catheter tube is closed at its proximal end and is formed as a conical rounded-off tip.

6. The device as set forth in claim 3, wherein the first guide wire is passed through a hemostatic valve disposed at a distal end of the catheter tube.

7. The device as set forth in claim 1, wherein the second guide wire has a length of about 10 to 30 cm.

8. The device as set forth in claim 1, wherein the fixing point is about 20 to 40 cm from the proximal end of the first guide wire.

9. The device as set forth in claim 4, wherein the ends of the first and second guide wires are curved with a radius of about 5 to 20 mm.
